(19) United States
(12) Patent Application Publication
(10) Pub. No.: US 2025/0166760 A1
(43) Pub. Date: May 22, 2025

(54) Information Tag and Emergency Medical Wristband

(71) Applicant: Lauren Crafton, Mooresville, NC (US)

(72) Inventor: Lauren Crafton, Mooresville, NC (US)

(21) Appl. No.: 18/512,325

(22) Filed: Nov. 17, 2023

(51) Int. Cl. G16H 10/65

(52) U.S. Cl. CPC G16H 10/65

(57) ABSTRACT

An information tag can be affixed to a product used by an infant, young child, or other person needing medical attention. The information tag includes a removable strip containing emergency information. In the event of an emergency, the removable strip can be detached from the tag and used as an emergency medical wristband.

### TECHNICAL FIELD

The present disclosure relates generally to information tags for emergency medical use and, more particularly, to an information tag that can be affixed to a product associated with a child or other person for providing emergency medical information to first responders in the event of an emergency.

### BACKGROUND

In emergency situations, an infant, young child, elderly person, or a person with certain disabilities may be unable to provide vital information to first responders, which may be required to provide proper care for the person. A parent or guardian for the person typically provides information required by first responders but, in many cases, the parent or guardian is unavailable. In case of a motor vehicle accident or mass casualty event, the parent or guardian may be incapacitated or become separated from the person under their care. In such cases, the first responders attending to the child or other person needing medical attention have little or no information about the person requiring treatment.

### SUMMARY

The present disclosure relates to an information tag that can be affixed to a product used by an infant, young child, or other person under the care of a parent or guardian. As one example, the information tag may be affixed to a child safety seat. The information tag includes a removable strip containing emergency information. In some embodiments, the removable strip includes a printed barcode encoded with information for accessing an online database storing personal and/or medical information about the person. In the event of an emergency, the removable strip can be detached from the tag and used as an emergency medical wristband.

### BRIEF DESCRIPTION OF THE DRAWINGS

FIG. 1 is a plan view showing the outer side of an information tag with a removable strip that can be separated from the tag and used as a wristband.

FIG. 2 is a plan view showing the inner side of the information tag.

FIG. 3 is a perspective view showing the information tag affixed to a car seat for an infant.

FIG. 4 is a perspective view showing the removable strip separated from the information tag and secured around the wrist of a person to form a wristband.

FIG. 5 illustrates a process for using an information tag with a printed barcode.

### DETAILED DESCRIPTION

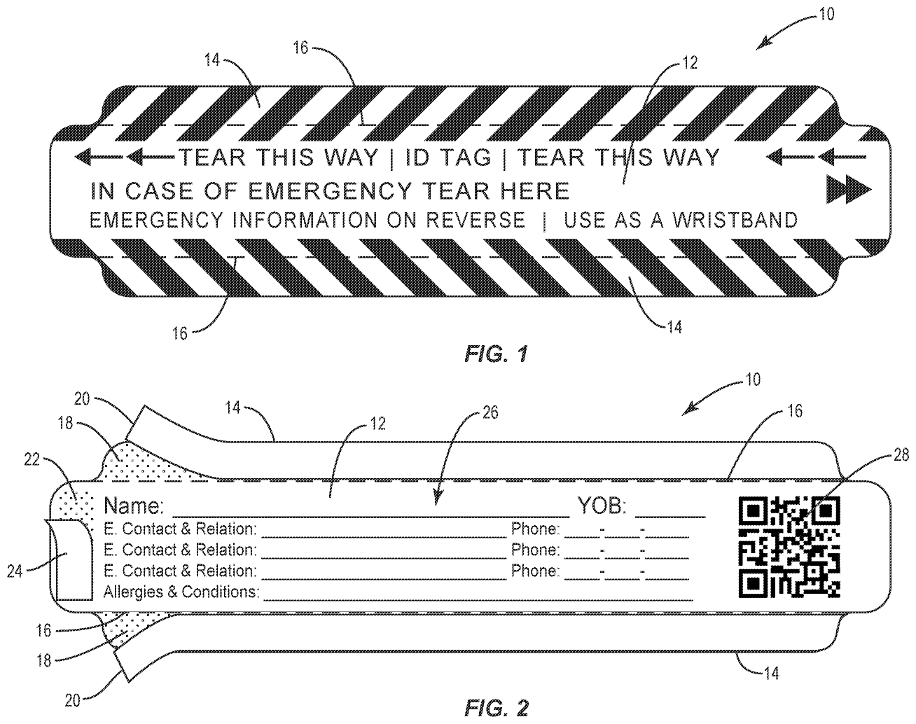

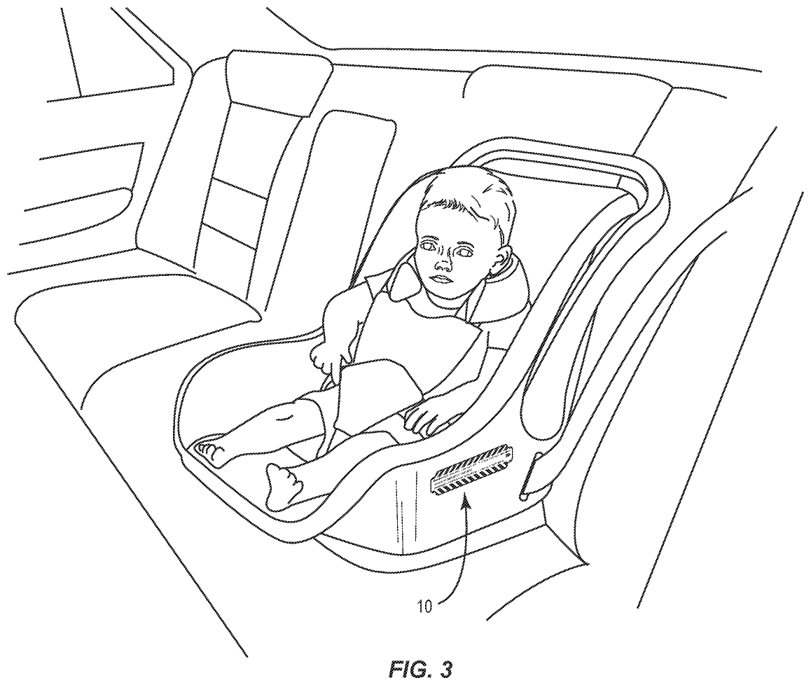

Referring now to the drawings, FIGS. 1 and 2 show an information tag 10 according to one exemplary embodiment. The information tag 10 can be affixed to a product used by an infant, young child, or other person requiring assistance. As one example, the information tag may be affixed to a child safety seat for an infant or young child. The information tag 10 includes a removable elongate strip 12 containing personal and/or medical information about a person. In the event of an emergency, the removable strip 12 can be torn off and used as an emergency medical wristband for the person.

The information tag 10 generally comprises an elongate strip 12 with side tabs 14 extending along opposing sides of the elongate strip 12. Perforated tear lines 16 separate the side tabs 14 from the elongate strip 12 and enable the elongate strip 12 to be torn off in an emergency. In the disclosed embodiment, the elongate strip 12 is longer than the side tabs 14 so that the elongate strip 12 protrudes from each end of the information tag 10. The protruding ends can be easily grasped by first responders in an emergency to tear off the elongate strip 12.

The information tag 10 can be affixed to a product designed for use by an infant, child, or other person requiring assistance. As shown in FIG. 2, an adhesive 18 is applied to the inner surfaces of the side tabs 14 for adhering the information tag 10 to a child safety seat or other product associated with the person. The adhesive 18 is covered by a protective cover 20 that can be peeled off and discarded when the information tag 10 is being affixed to the product.

FIG. 3 shows the information tag 10 affixed to a child safety seat for an infant or young child. The information tag 10 could also be affixed to a stroller, wheelchair, backpack, lunch box, or article of clothing.

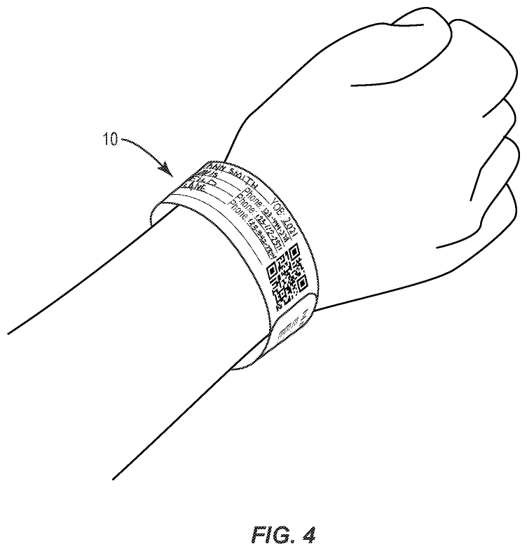

The elongate strip 12 is designed to be torn off in an emergency and secured around the wrist of an infant, young child, or other person requiring assistance as shown in FIG. 4. For securing the elongate strip 12 around a person's wrist, a second adhesive 22 (FIG. 2) is applied to the inner surface of the elongate strip 12 adjacent one end and is covered by a protective cover 24. When the elongate strip 12 is torn off, the protective cover 24 over the adhesive 22 is removed and the elongate strip 12 is wrapped around the persons' wrist to form the wristband. The wristband is secured in place by overlapping the ends of the elongate strip 12 with the adhesive 22 facing the overlapping end and pressing them together.

The inner surface of the elongate strip 12 includes an information region 26 containing placeholders for personal and/or medical information. In this example, the elongate strip 12 includes placeholders for the person's name, age or year of birth (YOB), contact information for up to three emergency contacts, and allergies or medical conditions. This information should preferably appear on the outside of the “wristband” as shown in FIG. 4.

In some embodiments, the inner surface of the elongate strip 12 may further include a printed barcode 28 containing relevant information for first responders in an emergency. For example, the barcode can be encoded with personal information identifying the person, contact information for a parent or guardian, and/or medical information. An application on a smartphone can be programmed to scan, decode, and display the information encoded into the barcode 28. Examples of information include a person's name and address, blood type, allergies, and/or other medical conditions that would be helpful to a first responder in an emergency.

In some embodiments, the barcode 28 is encoded with a uniform resource identifier (URI) that provides a link to medical information stored, for example, in an online database. In this case, an application on a smartphone or tablet can be programmed to scan and decode the URI, access the resource (e.g., online database) pointed to by the URI, and display information retrieved from the online database. Generally, this approach enables more information to be made available to the first responder.

In some embodiments, a bold pattern, such as contrasting stripes, is printed on the outer surface of the information tag 10 as shown in FIG. 1 along with instructions 18 for use in case of an emergency. In emergency situations, the emergency responder should not waste time looking for information. The bold pattern is designed to catch the attention an emergency responder. Emergency personnel can be trained to look for the information tag 10 when providing care in critical situations, as the information they contain can significantly impact the treatment and outcomes for the person requiring care.

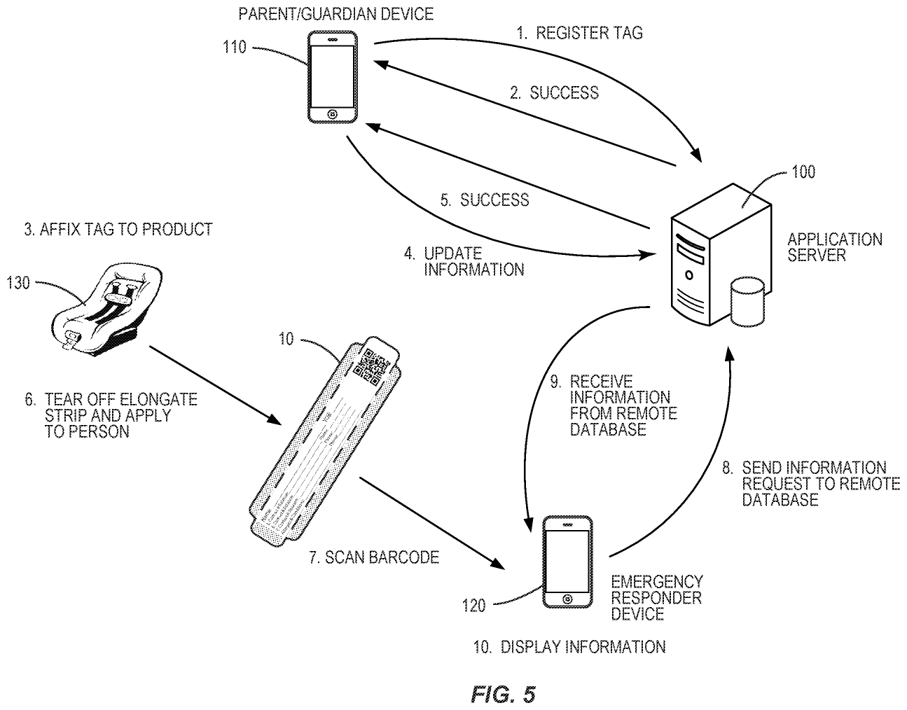

FIG. 5 illustrates an exemplary process for using an information tag 10 with a printed barcode 28. Prior to use, the parent or guardian registers the barcode with an application server 100 using a computing device 110 (step 1). The application server 100 includes a database for storing personal and/or medical information linked to printed barcodes 28. During registration, a barcode identifier is sent to the application server 100 and a form is presented to the user on the display of the computing device 110 to enter personal/medical information for a child or other person under the care of the parent/guardian. During the registration process, the parent/guardian may provide a username and password that can be later used to update the information. When the registration process is completed, the user is notified that the registration was successful (step 2).

In some embodiments, the application server 100 includes a web interface enabling the parent/guardian to access and communicate with the application server 100 using a standard web browser. To access the application server 100, the parent/guardian enters the URI of the application server 100 in the address bar of a browser to access a web application running on the application server 100. The barcode identifier and URI may be included in the instructions or packaging accompanying the information tag 10. The web application may present the parent/guardian with a series of forms for entering relevant information.

In some embodiments, a client application may be downloaded and installed on a smartphone or tablet of the parent/guardian. The client side application can allow the parent/guardian to scan the barcode 28 on the information tag 10 or packaging using a built-in camera on the smartphone or tablet. The client application can automatically extract the bar code identifier and URI for accessing the remote database. The web application may present the parent/guardian with a series of forms for entering relevant information.

Once the registration process is completed, the parent/guardian can affix the information tag 10 to a product 130, such as a child safety seat, stroller, wheelchair, or other product used by the person under the care of the parent/guardian (step 3). To affix the information tag 10 to the product 130, the protective covers 20 on the side tabs 14 are removed and the information tag 10 is applied face-down to the product 130, i.e., with the personal/medical information towards the product. In some embodiments, the parent/guardian writes or prints personal or medical information in the information region 26 of the information tag 10 prior to affixing the information tag 10 to a product 130. In this case, the information will be concealed from view until the elongate strip 12 is torn off or separated.

After the information tag 10 is affixed to the product 130, the parent guardian may access the application server 100 to update the information (step 4). When accessing the application server 100, the parent/guardian will be prompted to enter the credentials (i.e., username and password) entered during the registration process before being allowed to update the information. When the update process is completed, the user is notified that the information was successfully updated (step 5).

In the event of an emergency where the parent/guardian is unavailable, an emergency responder can tear off the elongate strip 12 on the information tag 10 to reveal the information on the inner surface (step 6). Any information contained in the information region will be immediately available to the emergency responder.

Additionally, the emergency responder can use a computing device 120 equipped with a scanner or camera to read and decode the information contained in the barcode 28 to obtain additional information. In one embodiment, the encoded information includes the barcode ID and URI for accessing the application server 100, which can be scanned by the emergency responder (step 7). With this information, the emergency responder can access the application server 100 using a computing device 120 having a browser or client application for that purpose. The application on the computing device (e.g. browser or client application) sends an information request to the application server 100 (step 8). The information request includes the barcode ID obtained by scanning the information tag 10. In response to the information request, the application server returns personal and/or medical information associated with the barcode ID contained in the information request (step 9). In some embodiments, the emergency responder may be required to identify themselves or provide credentials to access the application server 100. After receiving the information, the application on the computing device 12 displays the information on the display of the computing device 120 (step 10).

The information tag 10 is designed to provide vital information to emergency responders, healthcare professionals, and others in the event of a medical emergency when the person being treated is unable to communicate their medical history or needs. The information tag 10 is particularly useful for people who are non-verbal, unconscious, or unable to communicate effectively due to their age or condition. The information made available to emergency responders via the information tag 10 can include:
Personal Information: The person's name, address, and date of birth can be provided for quickly identifying the individual.
Medical Conditions: Information concerning medical conditions, chronic illnesses, or specific health issues can be provided.
Allergies: Any known allergies to medications, foods, or other substances can be provided to prevent adverse reactions.
Medications: Lists of prescription medications and dosages can be provided to ensure proper treatment and to avoid potential drug interactions.
Emergency Contacts: Contact information for family members, caregivers, or designated emergency contacts.
Blood Type: The person's blood type can be provided, which can be crucial in emergencies involving blood transfusions.
Other Information: Some embodiments may also include information on organ donor status or any advanced directives the individual may have.

The present invention may, of course, be carried out in other specific ways than those herein set forth without departing from the spirit and essential characteristics of the invention. The present embodiments are, therefore, to be considered in all respects as illustrative and not restrictive, and all changes coming within the meaning and equivalency range of the appended claims are intended to be embraced therein.

What is claimed is:

1. An information tag comprising:
an attachment portion having a first adhesive applied to an inner surface thereof for affixing the information tag to a product associated with a person;
a removable elongate strip connected to the attachment portion and configured to form a wristband that can be secured around the person's wrist;
a second adhesive applied to a first end of the elongate strip for securing the elongate strip around the person's wrist with the first end overlapping a second end to form the wristband.

2. The information tag of claim 1, wherein the attachment portion comprises a pair of side tabs extending along opposing sides of the elongate strip.

3. The information tag of claim 2, wherein the elongate strip and side tabs are divided by perforation lines to enable the elongate strip of the tag to be torn away from the side tabs of the tag for use as a wristband.

4. The information tag of claim 1, further comprising an information region on an inside surface of the elongate strip for recording information about the person.

5. The information tag of claim 4, wherein the inside surface of the tag faces the product when the tag is adhered to the product so that the information region is concealed.

6. The information tag of claim 4, further comprising a bar code on an inside surface of the strip providing a link to a resource containing information about the person.

7. The information tag of claim 6, wherein the resource comprises a web page or database.

8. The information tag of claim 1, further comprising a removable protective cover applied over the first adhesive, wherein the protective cover is removed when the tag is affixed to a product.

9. A child carrier comprising:
a seat configured to receive a child; and
an information tag affixed to the seat, the information tag including an attachment portion affixing the information tag to the seat and a removable elongate strip configured to form a wristband that can be secured around the child's wrist when separated from the attachment portion.

10. The child carrier of claim 9, wherein the attachment portion comprises side tabs disposed on opposing sides of the elongate strip, wherein the elongate strip and side tabs are divided by perforation lines to enable the elongate strip of the tag to be torn away from the side tabs of the tag for use as a wristband.

11. The child carrier of claim 9, wherein the information tag further comprises a first adhesive applied to inner surfaces of the tabs for affixing the tag to the seat while allowing separation of the elongate strip from the tabs after the tag is affixed to the seat; and

12. The child carrier of claim 9, wherein the tag further comprises a second adhesive applied to a first end of the strip for securing the elongate strip around the person's wrist with the first end overlapping a second end to form the wristband.

13. The child carrier of claim 9, further comprising an information region on an inside surface of the strip for recording information about the person.

14. The child carrier of claim 13, wherein the inside surface of the tag faces the product when the tag is adhered to the product so that the information region is concealed.

15. The child carrier of claim 13, further comprising a bar code on an inside surface of the strip providing a link to a resource containing information about the person.

16. The child carrier of claim 15, wherein the resource comprises a web page or database.

17. The combined information tag and wristband of claim 13, further comprising removable protective covers applied over the first adhesive on the tab, wherein the protective covers are removed when the tag is affixed to a product.

18. An emergency medical information system for providing medical information for use in an emergency, the system comprising:
a database storing emergency medical information about a person;
an information tag configured to be affixed to a product associated with a person, the information tag including an attachment portion for affixing the information tag to the product and a removable elongate strip configured to form a wristband that can be secured around the person's wrist when separated from the attachment portion; and
a barcode printed on the removable strip and encoded with access information for accessing the emergency medical information.

19. The emergency medical information system of claim 18, further comprising a computing device having an application for decoding the barcode and accessing the emergency medical information stored in the database.

20. The emergency medical information system of claim 19, wherein the computing device further comprises a display to display the emergency medical information.

21. The emergency medical information system of claim 18, wherein the attachment portion comprises a pair of side tabs extending along opposing sides of the elongate strip.

22. The emergency medical information system of claim 21, wherein the elongate strip and side tabs are divided by perforation lines to enable the elongate strip of the tag to be torn away from the tabs of the tag for use as a wristband.

23. The emergency medical information system of claim 22, wherein the tag further comprises a first adhesive applied to inner surfaces of the tabs for affixing the tag to an product while allowing separation of the strip from the tabs after the tag is affixed to the product; and

24. The emergency medical information system of claim 23, wherein the tag further comprises a second adhesive applied to a first end of the strip for securing the strip around a person's wrist with the first end overlapping a second end to form the wristband.

25. The emergency medical information system of claim 18, further comprising an information region on an inside surface of the elongate strip for recording information about the person.

26. The emergency medical information system of claim 25, wherein the inside surface of the tag faces the product when the tag is affixed to the product so that the information region is concealed from view.

27. The emergency medical information system of claim 25, further comprising removable protective covers applied over the first adhesive on the tab, wherein the protective covers are removed when the tag is affixed to a product.

28. The method of using an information tag comprising an attachment portion for affixing the information tag to a product and a removable elongate strip configured to form a wristband that can be secured around the person's wrist, the method comprising:
separating an elongate strip from an information tag affixed to a product associated with a person, the elongate strip containing emergency information linked to the person that is revealed when the elongate strip is separated from the information tag; and
securing the elongate strip with the emergency information around the person's wrist to form an emergency medical wristband.

29. The method of claim 28, wherein the emergency information is concealed from view prior before the elongate strip is separated from the information tag.

30. The method of claim 28, wherein the emergency information comprises a barcode having a unique identifier and a uniform resource identifier linked to a resource containing personal and medical information of the person.

31. The method of claim 30, further comprising using the unique identifier and URI to obtain the personal and medical information of the user.
